(19) United States
(12) Patent Application Publication
(10) Pub. No.: US 2024/0164758 A1
(43) Pub. Date: May 23, 2024

(54) SYSTEMS AND METHODS FOR GENERATING PATIENT MODELS BASED ON ULTRASOUND IMAGES

(71) Applicant: Shanghai United Imaging Intelligence Co., Ltd., Shanghai (CN)

(72) Inventors: Ziyan Wu, Lexington, MA (US); Shanhui Sun, Lexington, MA (US); Arun Innanje, Lexington, MA (US); Benjamin Planche, Briarwood, NY (US); Abhishek Sharma, Boston, MA (US); Meng Zheng, Cambridge, MA (US)

(73) Assignee: Shanghai United Imaging Intelligence Co., Ltd., Shanghai (CN)

(21) Appl. No.: 17/989,251

(22) Filed: Nov. 17, 2022

(51) Int. Cl. A61B 8/08; A61B 6/00; A61B 8/00

(52) U.S. Cl. CPC A61B 8/5261; A61B 6/5247; A61B 8/4254; A61B 8/466; A61B 8/5223

(57) ABSTRACT

Sensing device(s) may be installed in a medical environment to captures images of the medical environment, which may include an ultrasound probe and a patient. The images may be processed to determine, automatically, the position of the ultrasound probe relative to the patient's body. Based on the determined position, ultrasound image(s) taken by the ultrasound probe may be aligned with a 3D patient model and displayed with the 3D patient model, for example, to track the movements of the ultrasound probe and/or provide a visual representation of the anatomical structure(s) captured in the ultrasound image(s) against the 3D patient model. The ultrasound images may also be used to reconstruct a 3D ultrasound model of the anatomical structure(s).

### BACKGROUND

Conventional patient modeling methods can only obtain a three-dimensional (3D) surface model of the patient. To enrich the information that may be encompassed in a patient model and make it applicable to more clinical applications, it may be desirable to model the interior anatomical structures of the patient together with the 3D surface model. Information regarding these interior anatomical structures may be obtained through one or more of the common medical scans such as computed tomography (CT), X-ray, magnetic resonance imaging (MRI), or ultrasound imaging. Compared to the other imaging techniques, ultrasound imaging may be faster and safer, have a noninvasive nature, and be less expensive than the MRI and CT alternatives. Accordingly, it may be beneficial to obtain the information regarding a patient's interior anatomical structures by using ultrasound imaging techniques.

### SUMMARY

Described herein are systems, methods and instrumentalities associated with generating a three-dimensional (3D) patient model based on ultrasound images of the patient. A system as described herein may comprise at least one sensing device and one or more processors communicatively coupled to the at least one sensing device. The at least one sensing device may be configured to capture images of the patient in a medical environment, wherein the medical environment may include an ultrasound machine with an ultrasound probe. The at least one sensing device may be installed on the ultrasound machine or on a ceiling of the medical environment, and may be configured to capture images of the medical environment. The one or more processors may be configured to obtain a 3D human model of the patient, wherein the 3D human model may indicate at least a pose and a shape of the patient's body. The one or more processors may be further configured to receive a first ultrasound image of the patient captured using the ultrasound probe, determine, based on the captured images of the medical environment, a position of the ultrasound probe (e.g., relative to the patient's body), and align the first ultrasound image with the 3D human model of the patient based at least on the position of the ultrasound probe. The one or more processors may then generate a visual representation that shows the alignment of the first ultrasound image and the 3D human model.

In one or more embodiments, the visual representation may include a 3D body contour of the patient, and the one or more processors may be configured to fill a first inside portion of the 3D body contour with the first ultrasound image based on the alignment of the first ultrasound image and the 3D human model of the patient. In one or more embodiments, the one or more processors may be further configured to receive a second ultrasound image of the patient captured using the ultrasound probe, align the second ultrasound image with the 3D human model based on at least the position of the ultrasound probe, and add the second ultrasound image to the visual representation by filling a second inside portion of the 3D body contour with the second ultrasound image based on the alignment of the second ultrasound image and the 3D human model of the patient. In one or more embodiments, the first and second ultrasound images of the patient may be associated with an anatomical structure (e.g., an internal organ such as the heart) of the patient, and the one or more processors may be further configured to reconstruct a 3D ultrasound model of the anatomical structure based on at least the first ultrasound image and the second ultrasound image.

In one or more embodiments, the 3D human model of the patient may be obtained from another source or generated by the one or more processors based on the images (e.g., of the medical environment) captured by the at least one sensing device. In one or more embodiments, the one or more processors may be further configured to determine an orientation of the ultrasound probe (e.g., relative to the patient's body), and align the first ultrasound image with the 3D human model further based on the determined orientation of the ultrasound probe. In one or more embodiments, the one or more processors being configured to determine the position of the ultrasound probe may include the one or more processors being configured to detect, in the images of the medical environment, a marker associated with the ultrasound probe and determine the position of the ultrasound probe relative to the patient's body based on the detected marker. Alternatively, or additionally, the one or more processors may be further configured to determine the position of the ultrasound probe by detecting, using a machine learning model, visual features associated with the ultrasound probe in the captured images of the medical environment and determining the position of the ultrasound probe based on the detected visual features.

In one or more embodiments, the one or more processors may be further configured to determine, based on respective visual features extracted by a machine learning model from multiple ultrasound images, that two or more of the ultrasound images are substantially similar to each other, and provide an indication that the two or more ultrasound images are duplicative. In one or more embodiments, the one or more processors may be further configured to detect, based on a machine learning model, a medical abnormality in an ultrasound image, and provide an indication of the detection. For example, the indication may include a bounding box around the detected medical abnormality in the ultrasound image.

### BRIEF DESCRIPTION OF THE DRAWINGS

A more detailed understanding of the examples disclosed herein may be had from the following description, given by way of example in conjunction with the accompanying drawing.

FIG. 1 is a block diagram illustrating an example system described herein that may be used to generate one or more models of a patient (e.g., depicting the body surface and/or one or more interior anatomical structures of the patient) based on ultrasound images of the patient, in accordance with one or more embodiments described herein.

FIGS. 2A and 2B are simplified diagrams illustrating an example user interface (UI) for aligning a first ultrasound image of an interior anatomical structures with a 3D human model, in accordance with one or more embodiments described herein.

FIGS. 3A and 3B are simplified diagrams illustrating an example UI for aligning a second ultrasound image of an interior anatomical structures with a 3D human model in accordance with one or more embodiments described herein.

FIG. 4 is a simplified flow diagram illustrating an example method that may be associated with the training of a neural network in accordance with one or more embodiments described herein.

FIG. 5 is a flow diagram illustrating an example method that may be performed for generating a three-dimensional (3D) human model for a patient based on ultrasound images of the patient in accordance with one or more embodiments described herein.

FIG. 6A is a flow diagram illustrating an example method that may be performed for modifying a visual representation of the 3D human model based on an additional ultrasound image of the patient in accordance with one or more embodiments described herein.

FIG. 6B is a flow diagram illustrating an example method that may be performed for generating a 3D ultrasound model of an interior anatomical structure of the patient in accordance with one or more embodiments described herein.

FIG. 7 a block diagram illustrating an example of a sensing device in accordance with one or more embodiments described herein.

FIG. 8 a block diagram illustrating an example of a processing device in accordance with one or more embodiments described herein.

### DETAILED DESCRIPTION

The present disclosure is illustrated by way of example, and not by way of limitation, in the figures of the accompanying drawings.

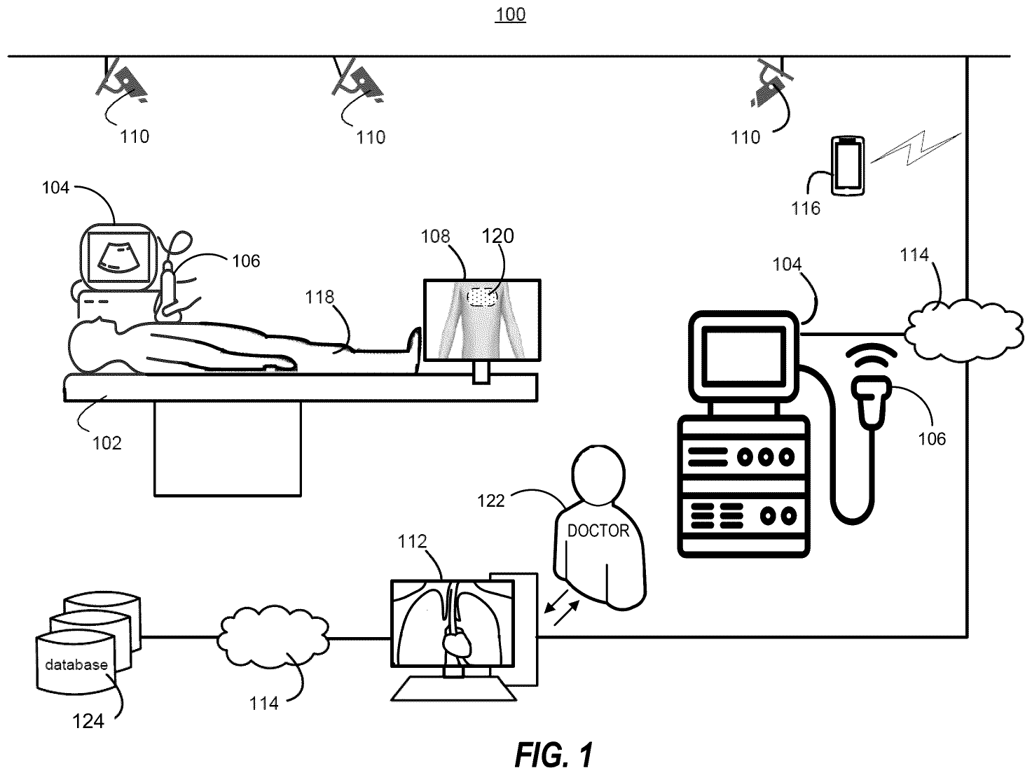

FIG. 1 is a block diagram illustrating an example system described herein that may be used to generate a three-dimensional (3D) human model for a patient 118 based on ultrasound images of the patient in a medical environment 100 in accordance with one or more embodiments described herein. The medical environment 100 may be any facility in a healthcare setting including, e.g., a scan room (e.g., magnetic resonance imaging (MRI), X-ray, Computed Tomography (CT), Ultrasound, etc.), an operating room (OR), a rehabilitation facility, etc. The medical environment 100 may be equipped with various tools, devices, and/or equipment such as a patient bed 102, an ultrasound machine 104 with an ultrasound probe 106, a patient monitoring device 108, etc. The tools, devices, and/or equipment may be maneuvered (e.g., manually or automatically) to accommodate the needs of a medical procedure being performed on patient 118 in the medical environment 100. For example, the patient bed 102 may be raised or lowered, the ultrasound probe 106 may be manipulated (e.g., moved, tilted, or rotated) towards a specific location (e.g., towards an internal anatomical structure 120), a lighting device (not shown) may be adjusted to focus on an ultrasound scan site, etc.

One or more sensing devices 110 may be installed at various locations of the medical environment 100 and may be communicatively coupled to a processing device 112 (e.g., comprising one or more processors) and/or other devices of the medical environment 100 via a communication network 114. Each of the sensing devices 110 may include one or more sensors such as one or more 2D visual sensors (e.g., 2D cameras), one or more 3D visual sensors (e.g., 3D cameras), one or more red, green and blue (RGB) sensors, one or more depth sensors, one or more RGB plus depth (RGB-D) sensors, one or more thermal sensors (e.g., infrared (FIR) or near-infrared (NIR) sensors), one or more motion sensors, one or more radar sensors, and/or other types of image capturing circuitry that are configured to capture images of a person, an object or a scene in the medical environment 100. Depending on the type of cameras, sensors, and/or image capturing circuitry included in the sensing devices 110, the images generated by the sensing devices 110 may include, for example, one or more photos, one or more thermal images, one or more radar images, and/or the like. The sensing devices 110 may be configured to generate the images described herein in response to detecting a person (e.g., patient 118), an object (e.g., ultrasound probe 106), or a scene (e.g., a standing medical professional, such as doctor 122, examining the patient 118 lying on the patient bed 102) in the medical environment 100. The sensing devices 110 may also be configured to generate the images described herein based on a preconfigured schedule or time interval, or upon receiving a control signal (e.g., from a remote control device like programming device 116) that triggers the image generation.

Each of the sensing devices 110 may include a functional unit (e.g., a processor) configured to control the image capturing functionalities described herein. The functional unit may also be configured to process the images (e.g., pre-process the images before sending the images to another processing device), communicate with other devices located inside or outside of the medical environment 100, determine a characteristic (e.g., a person or object) of the medical environment 100 based on the captured images, etc. For example, the functional unit (and/or the processing device 112) may be capable of generating (e.g., constructing) a 3D human model such as a 3D human mesh model of the patient 118 (e.g., a 3D patient model) based on the images captured by the sensing devices 110. Such a 3D human model may include a plurality of parameters that may indicate the body shape and/or pose of the patient while the patient is inside medical environment 100 (e.g., during an MRI, X-ray, Ultrasound, or CT procedure). For example, the parameters may include shape parameters β and pose parameters θ that may be used to determine multiple vertices (e.g., 6890 vertices based on 82 shape and pose parameters) associated with the patient's body and construct a visual representation of the patient model (e.g., a 3D mesh), for example, by connecting the vertices with edges to form polygons (e.g., such as a triangles), connecting multiple polygons to form a surface, using multiple surfaces to determine a 3D shape, and applying texture and/or shading to the surfaces and/or shapes.

The 3D patient model described above may also be generated by processing device 112. For example, processing device 112 may be communicatively coupled to one or more of sensing devices 110 and may be configured to receive images of the patient 118 from those sensing devices 110 (e.g., in real time or based on a predetermined schedule). Using the received images, processing device 112 may construct the 3D patient model, for example, in a similar manner as described above. It should be noted here that, even though processing device 112 is shown in FIG. 1 as being separate from sensing devices 110, any one of sensing devices 110 may be configured to operate as the processing device 112 (e.g., using one or more functional units or processors included in the sensing device 110). For example, sensing devices 110 may be inter-connected via communication network 114 as described below and exchange images with each other. One of the sensing devices 110 may be configured to perform the 3D patient model construction tasks described herein based on images received from the other sensing device(s) 110.

As noted above, each of the sensing devices 110 may include a communication circuit and may be configured to exchange information with one or more other sensing devices via the communication circuit and/or the communication network 114. The sensing devices 110 may form a sensor network within which the sensing devices 110 may transmit data to and receive data from each other. The data exchanged between the sensing devices 110 may include, for example, imagery data captured by each sensing device 110 and/or control data for discovering each sensing device's 110 presence and/or calibrating each sensing device's 110 parameters. For instance, when a new sensing device 110 is added to the medical environment 100, the sensing device 110 may transmit messages (e.g., via broadcast, groupcast or unicast) to one or more other sensing devices 110 in the sensor network and/or a controller (e.g., a processing device as described herein) of the sensor network to announce the addition of the new sensing device 110. Responsive to such an announcement or to a transmission of data, the other sensing devices 110 and/or the controller may register the new sensing device 110 and begin exchanging data with the new sensing device 110.

The sensing devices 110 may be configured to be installed at various locations of the medical environment 100 including, e.g., on a ceiling, above a doorway, on a wall, on a medical device, etc. From these locations, each of the sensing devices 110 may capture images of a person, object or scene that is in the field of view (FOV) of the sensing device 110 (e.g., the FOV may be defined by a viewpoint and/or a viewing angle). The FOV of each of the sensing devices 110 may be adjusted manually or automatically (e.g., by transmitting a control signal to the sensing device) so that the sensing device 110 may take images of a person, an object, or a scene in the medical environment 100 from different viewpoints or different viewing angles.

Each of the sensing devices 110 may be configured to exchange information with other devices (e.g., ultrasound machine 104 or monitoring device 108) in the medical environment 100, e.g., via the communication network 114. The configuration and/or operation of the sensing devices 110 may be at least partially controlled by a programming device 116. For example, the programming device 116 may be configured to initialize and modify one or more operating parameters of the sensing devices 110 including, e.g., the resolution of images captured by the sensing devices 110, a periodicity of data exchange between the sensing devices 110 and the processing device 112, a frame or bit rate associated with the data exchange, a duration of data storage on the sensing devices, etc. The programming device 116 may also be configured to control one or more aspects of the operation of the sensing devices 110 such as triggering a calibration of the sensing devices 110, adjusting the respective orientations of the sensing devices 110, zooming in or zooming out on a person or object in the medical environment 100, triggering a reset, etc. The programming device 116 may be a mobile device (e.g., such a smartphone, a tablet, or a wearable device), a desktop computer, a laptop computer, etc., and may be configured to communicate with the sensing devices 110 and/or the processing device 110 over the communication network 114. The programming device 116 may receive information and/or instructions from a user (e.g., via a user interface implemented on the programming device 116) and forward the received information and/or instructions to the sensing devices 110 via the communication network 114.

The communication network 114 described herein may be a wired or a wireless network, or a combination thereof. For example, the communication network 114 may be established over a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) or 5G network), a frame relay network, a virtual private network (VPN), a satellite network, and/or a telephone network. The communication network 114 may include one or more network access points. For example, the communication network 114 may include wired and/or wireless network access points such as base stations and/or internet exchange points through which one or more devices in the medical environment 100 may be connected to exchange data and/or other information. Such exchange may utilize routers, hubs, switches, server computers, and/or any combination thereof.

The processing device 112 may be configured to receive images from the sensing devices 110 and determine one or more characteristics of the medical environment 100 based on the images. These characteristics may include, for example, people and/or objects that are present in the medical environment 100 and the respective locations of the people and/or objects in the medical environment 100. The people presented in the medical environment 100 may include, e.g., a patient 118 and/or medical staff (e.g., the doctor 122, a technician, a nurse, etc.) attending to the patient 118. The objects presented in the medical environment 100 may include, e.g., the ultrasound machine 104, the ultrasound probe 106, the monitoring device 108, the patient bed 102, and/or other medical devices or tools not shown in FIG. 1. Based on the determined characteristics of the medical environment 100, the processing device 112 may track the location of the ultrasound probe 106 with respect to the body of patient 118 to guide and/or automate one or more aspects of the operations inside the medical environment 100. For example, in response to determining the respective locations of the patient 118, the doctor 122, and the various medical devices (e.g., the ultrasound probe 106) in the medical environment 100 based on images of the medical environment captured by the sensing devices 110, the processing device 112 may generate the 3D human model of the patient 118 and automatically align a first ultrasound image (e.g., an image of the interior anatomical structure 120 captured using the ultrasound probe 106) with the 3D human model based at least on the position of the ultrasound probe 106 (e.g., relative to the body of patient 118). The respective locations of the patient 118, the doctor 122, and the various medical devices may include 3D locations (e.g., in terms of [X, Y, Z] coordinates) of the patient 118, the doctor 122, and the various medical devices in the medical environment 100.

Furthermore, the processing device 112 may be configured to automatically generate a 3D ultrasound model of an internal organ (e.g., anatomical structure 120) of the patient 118 based on multiple ultrasound images of the internal organ captured by the ultrasound probe 106 of ultrasound machine 104. The organ may be, for example, the spleen, liver, heart, etc. of the patient and the 3D ultrasound model of the internal organ may show, for example, the shape and/or location of the organ as it corresponds to the body of the patient as indicated by the 3D patient model. The operation of the ultrasound machine 104 may involve the doctor 122 moving the ultrasound probe 106 over the body of patient 118 around the area of the internal organ of interest (e.g., anatomical structure 120) to capture 2D ultrasound images of the organ. The captured 2D ultrasound images may be displayed on the screen output (e.g., a display of ultrasound machine 104 and/or the monitoring device 108). The 2D ultrasound images may show a cross-section of the internal organ, and the doctor 122 may then be able to estimate the health state of the internal organ based on the 2D ultrasound images.

In examples, the sensing devices 110 may be configured to capture images of the medical environment 100 that includes the patient 118, the ultrasound machine 104, and/or the ultrasound probe 106. The processing device 112 may be configured to obtain a 3D human model of the patient 118 (e.g., based on the images captured by the sensing devices 110 or from a different source such as a patient model database), wherein the 3D human model may indicate at least a pose and a shape of the body of the patient 118. A first ultrasound image of the patient 118 captured using the ultrasound probe 106 may be received by the processing device 112, which may determine, based on the captured images of the medical environment 100, a position of the ultrasound probe 106 (e.g., relative to the body of patient 118). The processing device 112 may then align the first ultrasound image with the 3D human model of the patient 118 based on at least the position of the ultrasound probe 106, and generate a visual representation that shows the alignment of the first ultrasound image and the 3D human model.

In examples, the visual representation may include a 3D body contour of the patient 118, and the processing device 112 may be configured to fill a first inside portion of the 3D body contour with the first ultrasound image based on the alignment of the first ultrasound image and the 3D human model of the patient 118. For example, if the first ultrasound image is a left-side view of the patient's stomach, the image may be displayed inside the 3D body contour, in an area that corresponds to the left side of the stomach. Furthermore, the processing device 112 may also be configured to receive a second ultrasound image of the patient 118 captured using the ultrasound probe 106 and align the second ultrasound image with the 3D human model based, at least, on the position of the ultrasound probe 106 (e.g., relative to the body of patient 118). The second ultrasound image may then be added to the visual representation, for example, by filling a second inside portion of the 3D body contour with the second ultrasound image based on the alignment of the second ultrasound image and the 3D human model of the patient 118.

In examples, the first and second ultrasound images of the patient 118 may be associated with the anatomical structure 120 (e.g., an internal organ such as the heart) of the patient 118, and the processing device 112 may be further configured to reconstruct a 3D ultrasound model of the anatomical structure 120 based, at least, on the first ultrasound image, the second ultrasound image, and the position or location of the ultrasound probe 106 determined from the images of the medical environment 100. For instance, with the help of the sensing devices 110, the position/location of the ultrasound probe 106 may be tracked while the ultrasound images of the patient are taken. The position/location information may then be used to determine the respective 3D viewpoints of the 2D ultrasound images and to align and fuse the 2D ultrasound images into a 3D reconstructed view based on the determined 3D viewpoints.

In examples, the processing device 112 may be further configured to determine an orientation of the ultrasound probe 106 (e.g., relative to the body of patient 118), and align the first ultrasound image with the 3D human model further based on the determined orientation of the ultrasound probe. For example, if the orientation of the ultrasound probe 106 with respect to the body of patent 118 is 180° (e.g., the probe is upside down with respect to the head-feet axis of the body of patent 118), then the ultrasound images captured by the ultrasound probe 106 may be rotated accordingly in order to align them with the 3D human model.

In examples, the processing device 112 may be configured to determine the position of the ultrasound probe 106 (e.g., relative to the body of patient 118) by detecting, in the images of the medical environment 100, a marker associated with the ultrasound probe 106, and determining the position of the ultrasound probe 106 relative to the body of patient 118 based on the detected marker. Alternatively, or additionally, the processing device 112 may be further configured to determine the position of the ultrasound probe 106 (e.g., relative to the body of patient 118) based on detecting, using a machine learning model, visual features associated with the ultrasound probe 106 in the captured images of the medical environment 100, and determining the position of the ultrasound probe 106 based on the detected visual features.

In examples, the processing device 112 may be further configured to receive a second ultrasound image of the patient 118 captured using the ultrasound probe 106 and determine, based on respective visual features of the first ultrasound image and the second ultrasound image detected by a machine learning model, that the first ultrasound image is substantially similar to the second ultrasound image. An indication (e.g., a visual indication) that the first ultrasound image and the second ultrasound image are duplicative of each other may be provided (e.g., to the doctor 122). In examples, the processing device 112 may be further configured to detect, based on a machine learning model, a medical abnormality in an ultrasound image, and provide an indication of the detection (e.g., on monitoring device 108). For example, the indication may include a bounding shape (e.g., a bounding box or a bounding circle) around the detected medical abnormality in the ultrasound image.

In examples, the processing device 112 may be configured to present the 3D human model and the 3D ultrasound model of an internal anatomical structure on a display device (e.g., on monitoring device 108) by presenting a graphical representation of the surface of the patient's body together with a graphical representation of the internal anatomical structure of the patient on the display device. In examples, the processing device 112 may be communicatively coupled to the database 124, for example, via the communication network 114. The database 124 may comprise a patient record repository that stores basic information of the patient 118, diagnostic and/or treatment histories of the patient 118, scan images of the patient 118, etc. As a part of the generation of the 3D human model based on ultrasound images of the patient 118, the processing device 112 may be configured to retrieve all or a subset of the medical records of the patient 118 from the database 124, analyze the retrieved medical records in conjunction with other information of the patient 118 gathered or determined by the processing device 112 (e.g., such as the 3D human model described herein), and generate the 3D human model and the 3D ultrasound model of an internal anatomical structure of the patient 118 based, at least in part, on the retrieved medical records. For example, based on past medical scans of the patient 118, body geometry of the patient 118, and/or other preferences and/or constraints associated with the patient 118, the processing device 112 may automatically determine the parameters and/or configurations of a device (e.g., the position and/or orientation of the ultrasound probe 106) used in the medical procedure and cause the parameters and/or configurations to be implemented for the medical device, e.g., by transmitting the parameters and/or configurations to a display device visible to the doctor 122. The processing device 112 may also display, for example, a medical scan associated with the anatomical structure 120 on a display (e.g., as requested by the doctor 122 via an interface of the processing device 112) in order to assist the doctor 122.

In the examples, one or more of the tasks are described as being initiated and/or implemented by a processing device, such as the processing device 112, in a centralized manner. It should be noted, however, that the tasks may also be distributed among multiple processing devices (e.g., interconnected via the communication network 114, arranged in a cloud-computing environment, etc.) and performed in a distributed manner. Further, even though the processing device 112 has been described herein as a device separate from the sensing devices (e.g., the sensing devices 110), the functionalities of the processing device 112 may be realized via one or more of the sensing devices (e.g., the one or more sensing devices 110 may comprise respective processors configured to perform the functions of the processing device 112 described herein). Therefore, in some implementations, a separate processing device may not be included and one or more sensing devices (e.g., the sensing devices 110) may assume the responsibilities of the processing device.

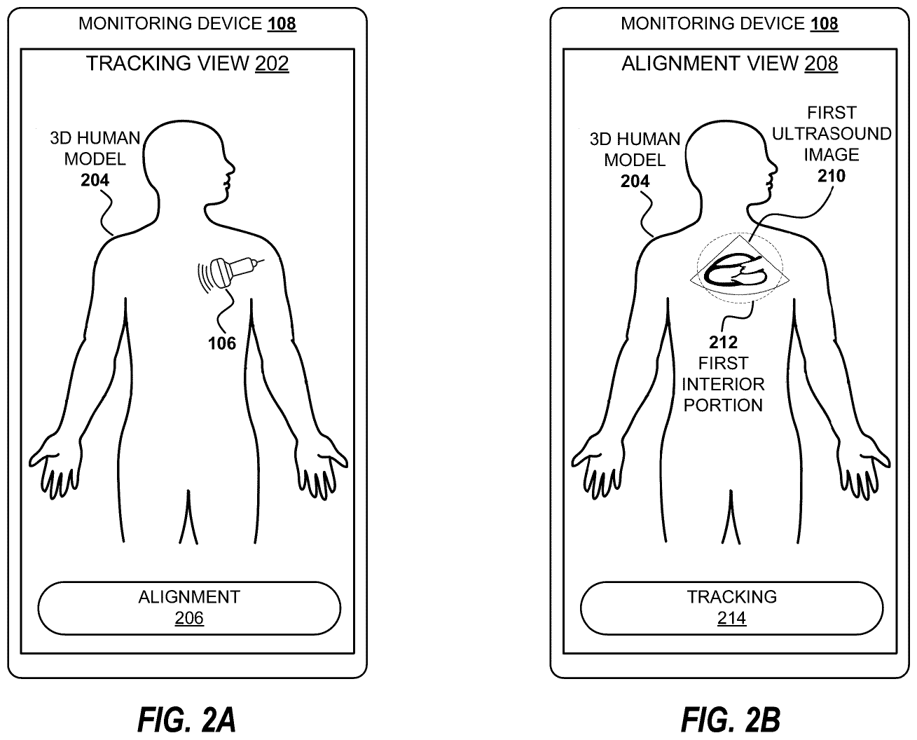

FIG. 2A and FIG. 2B are simplified diagrams illustrating an example user interface (UI) for aligning a first ultrasound image 210 of an interior anatomical structure (e.g., anatomical structure 120 in FIG. 1, such as the heart) with a 3D human model 204 in accordance with one or more embodiments described herein. The 3D human model 204 may be a generic patient model (e.g., a generic 3D human mesh) or it may be a model specific to the patient, e.g., constructed based on images of the patient 118 captured by the sensing devices described herein. As shown in FIG. 2A, a display device (e.g., monitoring device 108 of FIG. 1) may display a “tracking view” screen 202 that may include a graphical representation of the 3D human model 204 as well as a graphical representation of an ultrasound probe (e.g., the ultrasound probe 106 of FIG. 1) as the ultrasound probe is positioned at a first location of the patient's body (e.g., in real time). A medical professional (e.g., the doctor 122 of FIG. 1) may use the tracking view screen 202 of FIG. 2A to confirm the position and/or orientation of the ultrasound probe 106 (e.g., with respect to the patient's body) based on the graphical representations of the ultrasound probe 106 and the 3D human model 204 as shown in the tracking view screen 202.

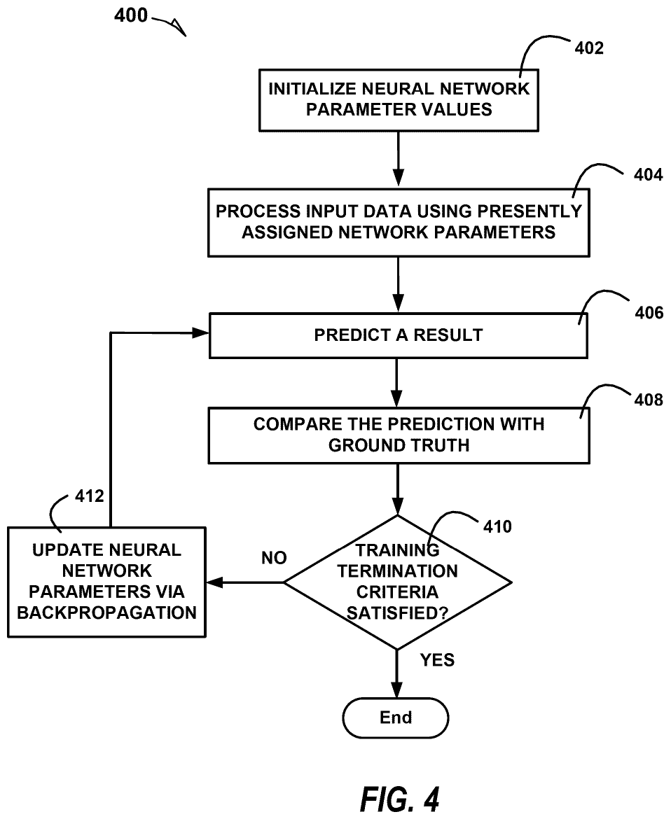

The tracking of the ultrasound probe 106 with respect to the patient's body may be accomplished based on images of the medical environment captured by the sensing devices described herein (e.g., the sensing devices 110 of FIG. 1). As described herein, these images may include a depiction of the ultrasound probe 106 and the patient, and therefore may be used to automatically determine the position and/or orientation of the ultrasound probe 106 (e.g., relative to the patient's body). In some embodiments, the ultrasound probe 106 may be identified in the images based on markers placed on the ultrasound probe 106. Alternatively or additionally, visual features of the ultrasound probe 106 may be learned using a machine learning (ML) model such that, when given the images that include the probe, the ML model may be used to predict the location of the ultrasound probe 106, as described further below with respect to FIG. 4.

In some embodiments, the tracking view screen 202 of FIG. 2A may include an “alignment” button 206 that may be used to activate and/or display an “alignment view” screen 208 in which a first ultrasound image 210 of the patient may be aligned with the 3D human model 204 based on the tracked location of the ultrasound probe 106 (e.g., the location of the ultrasound probe from which the first ultrasound image 210 is captured). Alternatively or additionally, the alignment view screen 208 may be activated and/or displayed independently from the tracking view screen 202, for example, without using the alignment button 206. In some embodiments, the alignment view screen 208 may display the first ultrasound image 210 within a first interior portion 212 of the 3D human model 204, where the first interior portion 212 may encompass the location(s) of the anatomical structure(s) captured by the first ultrasound image 210 inside the patient's body. A user (e.g., doctor 122 of FIG. 1) may then interact with the alignment view screen 208 (e.g., zoom in, rotate, drag, etc.) in order to examine the anatomical structure(s) shown in the first ultrasound image 210 within the context of the 3D human model 204.

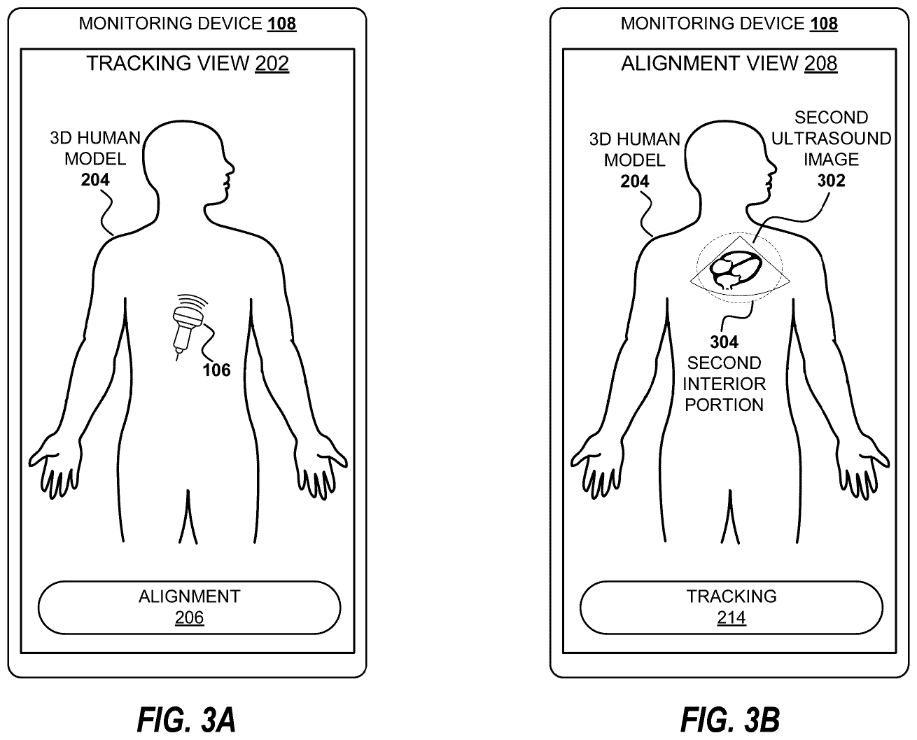

FIG. 3A and FIG. 3B are simplified diagrams illustrating an example UI for aligning a second ultrasound image 302 of an interior anatomical structure (e.g., anatomical structure 120 of patient 118 in FIG. 1, such as the heart) with the 3D human model 204 in accordance with one or more embodiments described herein. As shown in FIG. 3A, the display device (e.g., monitoring device 108 of FIG. 1) may display the tracking view screen 202 as part of the UI for aligning the second ultrasound image 302 with the 3D human model 204. The tracking view screen 202 of FIG. 3A may show the graphical representation of the 3D human model 204 of the patient's body as well as the graphical representation of the ultrasound probe 106 as the ultrasound probe is positioned at a second location of the patient's body (e.g., in real time). As noted above, a medical professional may use the tracking view screen 202 of FIG. 3A in order to confirm the position and/or orientation of the ultrasound probe 106 with respect to the patient's body based on the relative positions of the graphical representations of the ultrasound probe 106 and the 3D human model 204 as shown in the tracking view screen 202.

As noted above, the tracking view screen 202 of FIG. 3A may include the alignment button 206 for viewing a second ultrasound image 302 of the patient aligned with the graphical representation of the 3D human model 204 in the alignment view screen 208 based on the current tracked location of the ultrasound probe 106 being shown in the tracking view screen 202 of FIG. 3A. The alignment view screen 208 may also be activated/displayed independently from the tracking view screen 202, for example, without using the alignment button 206. As shown in FIG. 3B, the graphical representation of the 3D human model 204 may be displayed in the alignment view screen 208 together with the aligned second ultrasound image 302 based on the tracked position of the ultrasound probe 106 (e.g., relative to the patient's body) when the second ultrasound image 302 was captured. For example, the alignment view screen 208 may display the second ultrasound image 302 within a second interior portion 304 of the 3D human model 204, where the second interior portion 304 may encompass the location(s) of the anatomical structure(s) captured by the second ultrasound image 210 inside the patient's body. A user (e.g., doctor 122 of FIG. 1) may then interact with the alignment view screen 208 (e.g., zoom in, rotate, drag, etc.) in order to examine the anatomical structure(s) shown in the second ultrasound image 302 within the context of the 3D human model 204.

In some embodiments, multiple 2D ultrasound images (e.g., including the first ultrasound image 210 and the second ultrasound image 302) that have been aligned with the 3D human model 204 may be used to generate a 3D ultrasound model (e.g., as described herein), which may be displayed together with the graphical representation of the 3D human model 204 (e.g., within a third interior portion of the 3D human model 204) in the alignment view screen 208.

One or more of the tasks described herein (e.g., such as automatically recognizing an ultrasound probe and determining the position of the ultrasound probe) may be performed using an artificial neural network (e.g., based on a machining learning model implemented via the artificial neural network). In examples, such an artificial neural network may include a plurality of layers such as one or more convolution layers, one or more pooling layers, and/or one or more fully connected layers. Each of the convolution layers may include a plurality of convolution kernels or filters configured to extract features from an input image. The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map). In examples (e.g., if the task involves the generation of a segmentation mask associated with the ultrasound probe), the artificial neural network may further include one or more un-pooling layers and one or more transposed convolution layers that may be configured to up-sample and de-convolve the features extracted through the operations described above. As a result of the up-sampling and de-convolution, a dense feature representation (e.g., a dense feature map) of the input image may be derived, and the artificial neural network may be configured to predict the location of the ultrasound probe based on the feature representation.

FIG. 4 illustrates an example method 400 for training an artificial neural network (e.g., a machine learning model implemented by the neural network) to perform one or more of the tasks described herein. As shown, the training process may include initializing the operating parameters of the neural network (e.g., weights associated with various layers of the neural network) at 302, for example, by sampling from a probability distribution or by copying the parameters of another neural network having a similar structure. The training process may further include processing an input (e.g., a picture of a medical environment) using presently assigned parameters of the neural network at 404, and making a prediction for a desired result (e.g., identification of an object in the input such as an ultrasound probe) at 406. The prediction result may then be compared to a ground truth at 408 to calculate a loss associated with the prediction based on a loss function such as an MSE, an L1 norm, an L2 norm, etc. The calculated loss may be used to determine, at 410, whether one or more training termination criteria are satisfied. For example, the training termination criteria may be determined to be satisfied if the loss is below a threshold value or if the change in the loss between two training iterations falls below a threshold value. If the determination at 410 is that the termination criteria are satisfied, the training may end; otherwise, the presently assigned network parameters may be adjusted at 412, for example, by backpropagating a gradient descent of the loss function through the network before the training returns to 406.

For simplicity of explanation, the training operations are depicted in FIG. 4 and described herein with a specific order. Furthermore, it should be noted that not all operations that may be included in the training process are depicted and described herein, and not all illustrated operations are required to be performed.

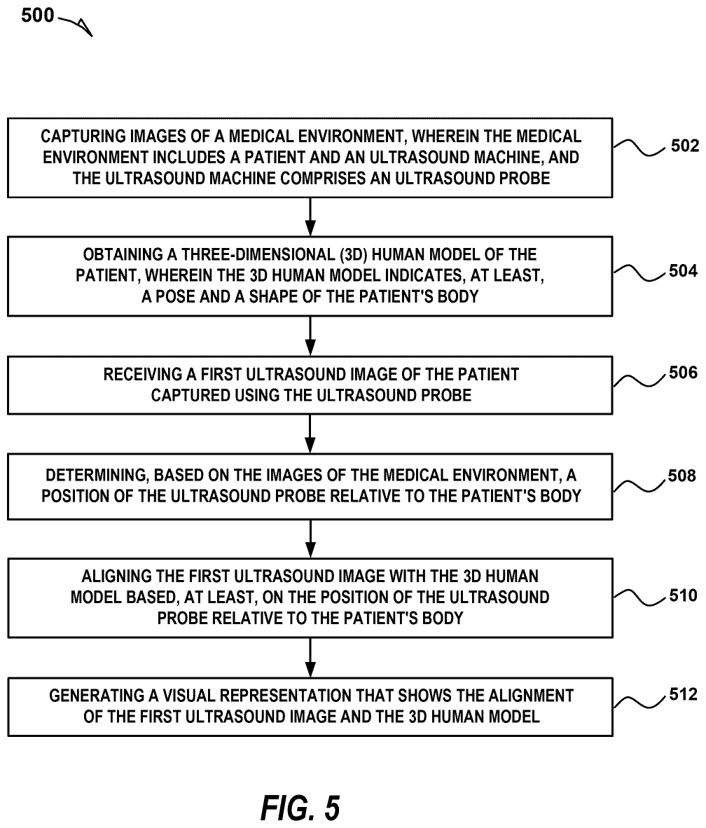

FIG. 5 is a flow diagram showing an example method 500 that may be performed by a processing device (e.g., the processing device 112) and/or one or more sensing devices (e.g., the sensing devices 110) to generate a 3D human model (e.g., 3D Human model 204 of FIGS. 2A-3B) based on ultrasound images (e.g., first ultrasound image 210 of FIG. 2B) of a patient (e.g., the patient 118) in a medical environment (e.g., the medical environment 100) in accordance with one or more embodiments described herein. The operations may start at 502, where images of the medical environment may be captured. The medical environment may include the patient and an ultrasound machine (e.g., ultrasound machine 104), and the ultrasound machine may include an ultrasound probe (e.g., ultrasound probe 106). The images may be captured, for example, by the one or more sensing devices 110 (e.g., which have been installed in the medical environment 100). At 504, the processing device may, e.g., based on a machine learning model, analyze the images, extract visual features from the images, and determine a patient model (e.g., a 3D parametric mesh) that may indicate, at least, a pose and a shape of the patient's body in the medical environment. Alternatively, the processing device may obtain the 3D human model of the patient from another source, such as the database 124 of FIG. 1. At 506, the processing device may receive a first ultrasound image of the patient captured using the ultrasound probe. For example, the ultrasound probe may be used to capture a first ultrasound image of an internal organ such as the heart (e.g., anatomical structure 120 of FIG. 1) of the patient.

At 508, the processing device may determine, based on the images of the medical environment, a position of the ultrasound probe (e.g., relative to the patient's body). For example, visual features associated with people (e.g., patient 118, doctor 124, etc.) and/or objects (e.g., ultrasound probe 106 or other tools, devices, etc.) in the images of the medical environment may be analyzed to determine respective locations of the persons and/or objects detected in the images in the medical environment and learn a spatial relationship of the persons or objects based on the determined locations. The processing device may assemble information from multiple images that may be captured by different sensing devices in order to determine the respective locations of a person and/or object. The processing device may accomplish this task by utilizing knowledge about the parameters of the sensing devices such as the relative positions of the sensing devices to each other and to the other people and/or objects in the medical environment. For example, the processing device may determine the depth (e.g., a Z coordinate) of a person or object in the medical environment based on two images captured by respective sensing devices, e.g., using a triangulation technique to determine the (X, Y, Z) coordinates of the person or object in the medical environment based on the camera parameters of the sensing device.

At 510, the processing device may align the first ultrasound image with the 3D human model based, at least, on the position of the ultrasound probe relative to the patient's body. For example, the processing device may determine that the ultrasound probe is positioned over the patient's chest area and therefore the captured first ultrasound image may be aligned with the 3D human model so that it is located at the chest area of the 3D human model of the patient. At 512, the processing device may generate a visual representation (e.g., on a display device) that shows the alignment of the first ultrasound image and the 3D human model. The processing device may continuously perform the operations of 502-508, for example, as new ultrasound images are captured for the patient.

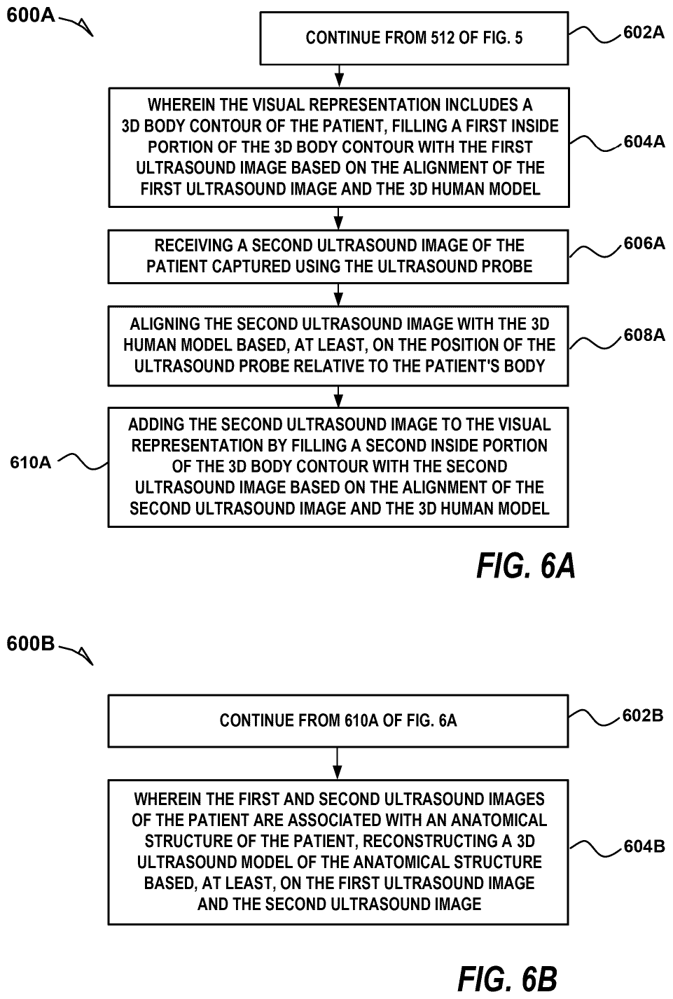

FIG. 6A a flow diagram illustrating an example method 600A that may be performed for modifying a visual representation of the 3D human model (e.g., 3D human model 204 of FIGS. 2A-3B) based on an additional ultrasound image of the patient (e.g., patient 118 of FIG. 1) in accordance with one or more embodiments described herein. The operations may start at 602A, as a continuation of operation 512 of method 500 of FIG. 5 described above. At 604A, a first inside portion of the 3D human model (e.g., a 3D body contour) may be filled with the first ultrasound image based on the alignment of the first ultrasound image and the 3D human model. For example, if the first ultrasound image is a right-side view of the patient's chest, the first ultrasound image may be displayed inside the 3D body contour, in an area that corresponds to the right side of the chest. At 606A, a second ultrasound image of the patient may be captured using the ultrasound probe (e.g., ultrasound probe 106 of FIG. 1), and the second ultrasound image may be aligned, at 608A, with the 3D human model based, at least, on the position of the ultrasound probe (e.g., relative to the patient's body). For example, if the ultrasound probe approaches the anatomical structure of the patient from a left-side of the chest, then the second ultrasound image may be aligned with the 3D human model based on these relative positons of the patient's body and the ultrasound probe. At 610A, the second ultrasound image may be added to the visual representation by filling a second inside portion of the 3D body contour with the second ultrasound image based on the alignment of the second ultrasound image and the 3D human model. For example, if the second ultrasound image is a top-side view of the patient's chest, the second ultrasound image may be displayed inside the 3D body contour, in an area that corresponds to the top side of the chest.

FIG. 6B a flow diagram illustrating an example method 600B that may be performed for generating a 3D ultrasound model of an interior anatomical structure (e.g., anatomical structure 120 of FIG. 1) of the patient (e.g., patient 118 of FIG. 1) in accordance with one or more embodiments described herein. The operations may start at 602B, as a continuation of operation 610A of method 600A of FIG. 6A described above. At 604B, the first and second ultrasound images of the patient associated with the anatomical structure of the patient may be used to reconstruct a 3D ultrasound model of the anatomical structure based, at least, on the first ultrasound image and the second ultrasound image. For example, a volume reconstruction method may be used to get 3D volume data associated with the anatomical structure which may then be used in a 3D volume grid based on spatial information acquired from visually tracking the ultrasound probe within the medical environment as explained above.

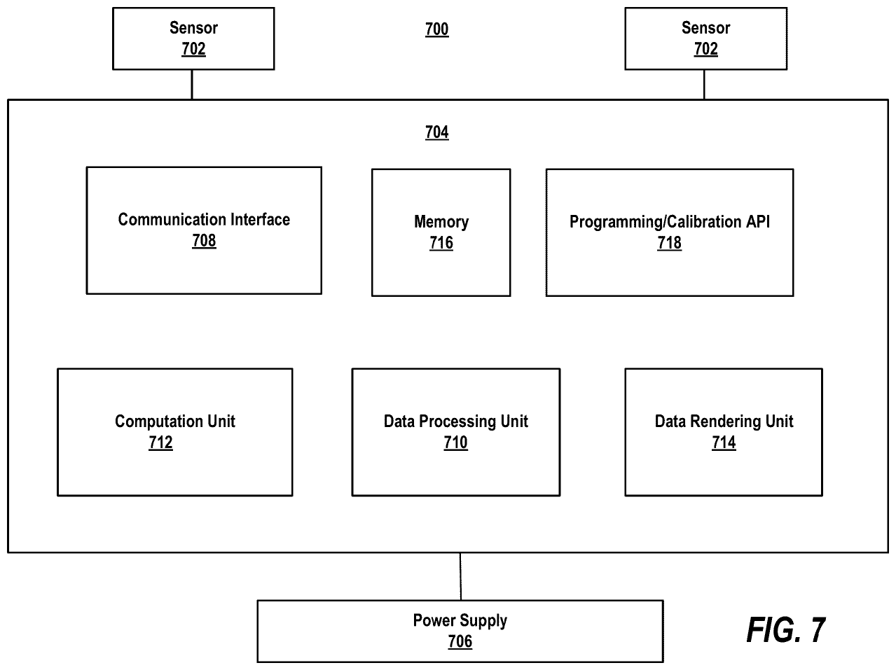

FIG. 7 illustrates an example sensing device 700 (e.g., the sensing devices 110 shown in FIG. 1) that may be placed or installed in a medical environment (e.g., the medical environment 100 of FIG. 1) to facilitate the generation of a 3D human model based on ultrasound images. The sensing device 700 may comprise a sensor 702, a functional unit 704, and/or a power supply 706 that may be configured to be hosted in a housing. Although two sensors are shown in the figure, the sensing device 700 may comprise any number of sensors. Further, although one or more of the components are shown in FIG. 7 as being inside or outside of the functional unit 704, these components may be moved out of or into the functional unit 704 without affecting the functionalities of the sensing device described herein.

As described herein, the sensor 702 may include a RGB sensor, a depth sensor, a RGB plus depth (RGB-D) sensor, a thermo-sensor such as a FIR or NIR sensor, a radar sensor, a motion sensor, a camera (e.g., a digital camera) and/or other types of image capturing circuitry configured to generate images (e.g., 2D images or photos) of a person, object, and/or scene in the FOV of the sensor. The images generated by the sensor 702 may include, for example, one or more photos, thermal images, and/or radar images of the person, object or scene. Each of the images may comprise a plurality of pixels that collectively represent a graphic view of the person, object or scene and that may be analyzed to extract features that are representative of one or more characteristics of the person, object or scene.

The sensor 702 may be communicatively coupled to the functional unit 704, for example, via a wired or wireless communication link. The sensor 702 may be configured to transmit images generated by the sensor to the functional unit 704 (e.g., via a push mechanism) or the functional unit 704 may be configured to retrieve images from the sensor 702 (e.g., via a pull mechanism). The transmission and/or retrieval may be performed on a periodic basis (e.g., based on a preconfigured schedule) or in response to receiving a control signal triggering the transmission or retrieval. The functional unit 704 may be configured to control the operation of the sensor 702. For example, the functional unit 704 may transmit a command to adjust the FOV of the sensor 702 (e.g., by manipulating a direction or orientation of the sensor 702). As another example, the functional unit 704 may transmit a command to change the resolution at which the sensor 702 takes images of a person, object or scene.

The sensor 702 and/or the functional unit 704 (e.g., one or more components of the functional unit 704) may be powered by the power supply 706, which may comprise an alternative current (AC) power source or a direct current (DC) power source (e.g., a battery power source). When a DC power source such as a battery power source is used, the power supply 706 may be rechargeable, for example, by receiving a charging current from an external source via a wired or wireless connection. For example, the charging current may be received by connecting the sensing device 700 to an AC outlet via a charging cable and/or a charging adaptor (including a USB adaptor). As another example, the charging current may be received wirelessly by placing the sensing device 700 into contact with a charging pad.

The functional unit 704 may comprise one or more of a communication interface circuit 708, a data processing device 710, a computation unit 712, a data rendering unit 714, a memory 716, or a programming and/or calibration application programming interface (API) 718. It should be noted that the components shown in FIG. 7 are provided merely as examples and are not meant to limit the scope of the disclosure. For example, the functional unit 704 is not restricted to including the exact components as shown in FIG. 7. Two or more of the components (e.g., functionalities of the components) may be combined, any one of the components may be divided into sub-components, any one of the components may be omitted, more components may be added, etc. As such, even though the functionalities of the sensing device 700 are described herein as being associated with respective one or more of the components, it will be appreciated that those functionalities may also be performed by a different component and/or be divided among multiple other components.

The functional unit 704 may be configured to receive or retrieve images from the sensor 702 via the communication interface circuit 708, which may include one or more wired and/or wireless network interface cards (NICs) such as ethernet cards, WiFi adaptors, mobile broadband devices (e.g., 4G/LTE/5G cards or chipsets), etc. In examples, a respective NIC may be designated to communicate with a respective sensor. In examples, a same NIC may be designated to communication with multiple sensors.

The images received or retrieved from the sensor 702 may be provided to the data processing device 710, which may be configured to analyze the images and carry out one or more of the operations described herein (e.g., including operations of the processing device 112 described herein). The functionality of the data processing device 710 may be facilitated by the computation unit 712, which may be configured to perform various computation intensive tasks such as feature extraction and/or feature classification based on the images produced by the sensor 702. The computation unit 712 may be configured to implement one or more neural networks. The data rendering unit 714 may be configured to generate the one or more visual representations described herein including, e.g., a representation of a 3D human model for a patient and a 3D ultrasound model of an anatomical structure of the patient, etc.

Each of the data processing device 710, the computation unit 712, or the data rendering unit 714 may comprise one or more processors such as a central processing device (CPU), a graphics processing device (GPU), a microcontroller, a reduced instruction set computer (RISC) processor, an application specific integrated circuit (ASIC), an application-specific instruction-set processor (ASIP), a physics processing device (PPU), a digital signal processor (DSP), a field programmable gate array (FPGA), or a combination thereof. The data processing device 710, computation unit 712, and/or data rendering unit 714 may also comprise other type(s) of circuits or processors capable of executing the functions described herein. Further, the data processing device 710, the computation unit 712, or the data rendering unit 714 may utilize the memory 716 to facilitate one or more of the operations described herein. For example, the memory 716 may include a machine-readable medium configured to store data and/or instructions that, when executed, cause the processing device 710, the computation unit 712, or the data rendering unit 714 to perform one or more of the functions described herein. Examples of a machine-readable medium may include volatile or non-volatile memory including but not limited to semiconductor memory (e.g., electrically programmable read-only memory (EPROM), electrically erasable programmable read-only memory (EEPROM)), flash memory, and/or the like. And even though not shown in FIG. 7, the sensing device 700 may also comprise one or more mass storage devices that include a magnetic disk such as an internal hard disk, a removable disk, a magneto-optical disk, a CD-ROM or DVD-ROM disk, etc., on which instructions and/or data may be stored to facilitate the performance of the functions described herein.

The operation of the sensing device 700 may be configured and/or controlled through the programming/calibration API 718, for example, using a remote programming device such as the programming device 116 in FIG. 1. In examples, the programming/calibration API 718 may be configured to receive commands (e.g., one or more digital messages) from the programming device that adjust the operating parameters of the sensing device 700 such as the orientation and/or FOV of a sensor, a resolution at which a sensor captures images, a periodicity at which images are received or retrieved from a sensor, etc. In response to receiving a command from the programming device, the sensing device 700 (e.g., the functional unit 704) may adjust one or more aspects of its operation in accordance with the command. For instance, if the command specifies a higher output quality, the sensing device 700 may output a high-resolution image in response, and if the command specifies a higher frame rate, the sensing device 700 may output lower-resolution images at increased frame rates.

The sensing device 700 (e.g., the functional unit 704) may also be configured to receive ad hoc commands through the programming/calibration API 718. Such ad hoc commands may include, for example, a command to zoom in or zoom out a sensor, a command to reset the sensing device 700 (e.g., restart the device or reset one or more operating parameters of the device to default values), a command to enable or disable a specific functionality of the sensing device 700, etc. The sensing device 700 (e.g., the functional unit 704) may also be programmed and/or trained (e.g., over a network) via the programming/calibration API 718. For example, the sensing device 700 may receive training data and/or operating logics through the programming/calibration API 718 during and/or after an initial configuration process.

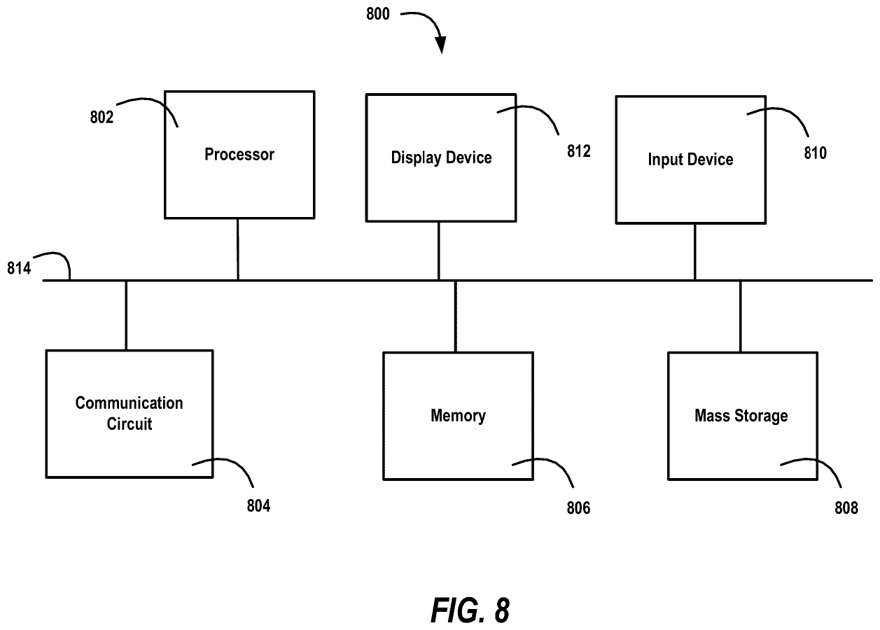

FIG. 8 illustrates example components of a processing device 800 (e.g., the processing device 112 of FIG. 1) as described herein. As shown, the processing device 800 may include a processor 802, which may be a central processing device (CPU), a graphics processing device (GPU), a microcontroller, a reduced instruction set computer (RISC) processor, application specific integrated circuits (ASICs), an application-specific instruction-set processor (ASIP), a physics processing device (PPU), a digital signal processor (DSP), a field programmable gate array (FPGA), or any other circuit or processor capable of executing the functions described herein. The processing device 800 may further include a communication circuit 804, a memory 806, a mass storage device 808, an input device 810, a display device 812, and/or a communication link 814 (e.g., a communication bus) over which the one or more components shown in FIG. 8 may exchange information. The communication circuit 804 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). The memory 806 may include a storage medium configured to store machine-readable instructions that, when executed, cause the processor 802 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. The mass storage device 808 may include one or more magnetic disks such as one or more internal hard disks, one or more removable disks, one or more magneto-optical disks, one or more CD-ROM or DVD-ROM disks, etc., on which instructions and/or data may be stored to facilitate the operation of the processor 802. The input device 810 may include a keyboard, a mouse, a voice-controlled input device, a touch sensitive input device (e.g., a touch screen), and/or the like for receiving user inputs to the processing device 800. The display device 812 may include one or more monitors (e.g., computer monitors, TV monitors, tablets, mobile devices such as smart phones, etc.), one or more speakers, one or more augmented reality (AR) devices (e.g., AR goggles), and/or other accessories configured to facilitate the visual representation of contents on the display device 812. These contents may include, for example, information generated by the processing device such as a 3D mesh of a patient, a 3D ultrasound model of an anatomical structure of the patient, a plot of radiation exposure over time, etc. The display may be rendered in various formats including, for example, videos, animations, and/or AR presentations.

It should be noted that the processing device 800 may operate as a standalone device or may be connected (e.g., networked or clustered) with other computation devices to perform the functions described herein. And even though only one instance of each component is shown in FIG. 8, a skilled person in the art will understand that the processing device 800 may include multiple instances of one or more of the components shown in the figure. Furthermore, although example operations of the processing device may be depicted and described herein in a specific order, the operations may also take place in other orders, concurrently, and/or with other operations not presented or described herein. Not all operations that the processing device is capable of performing are depicted and described herein, and not all illustrated operations are required to be performed by the processing device.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. A system, comprising:
at least one sensing device configured to capture images of a medical environment, wherein the medical environment includes a patient and an ultrasound machine, and the ultrasound machine comprises an ultrasound probe; and
one or more processors configured to:
obtain a three-dimensional (3D) human model of the patient, wherein the 3D human model indicates at least a pose and a shape of the patient's body;
receive a first ultrasound image of the patient captured using the ultrasound probe;
determine, based on the images of the medical environment captured by the at least one sensing device, a position of the ultrasound probe;
align the first ultrasound image with the 3D human model based on at least the position of the ultrasound probe; and
generate a visual representation that shows the alignment of the first ultrasound image and the 3D human model.

2. The system of claim 1, wherein the visual representation includes a 3D body contour of the patient, and wherein the one or more processors are further configured to fill a first inside portion of the 3D body contour with the first ultrasound image based on the alignment of the first ultrasound image and the 3D human model.

3. The system of claim 2, wherein the one or more processors are further configured to:
receive a second ultrasound image of the patient captured using the ultrasound probe;
align the second ultrasound image with the 3D human model based on at least the position of the ultrasound probe; and
add the second ultrasound image to the visual representation by filling a second inside portion of the 3D body contour with the second ultrasound image based on the alignment of the second ultrasound image and the 3D human model.

4. The system of claim 3, wherein the first and second ultrasound images of the patient are associated with an anatomical structure of the patient, and the one or more processors are further configured to reconstruct a 3D ultrasound model of the anatomical structure based on at least the first ultrasound image and the second ultrasound image.

5. The system of claim 1, wherein the 3D human model of the patient is obtained from another source or generated by the one or more processors based on the images captured by the at least one sensing device.

6. The system of claim 1, wherein the one or more processors are further configured to determine an orientation of the ultrasound probe, and align the first ultrasound image with the 3D human model further based on the determined orientation of the ultrasound probe.

7. The system of claim 1, wherein the one or more processors being configured to determine the position of the ultrasound probe comprises the one or more processors being configured to detect, in the images of the medical environment, a marker associated with the ultrasound probe and determine the position of the ultrasound probe based on the detected marker.

8. The system of claim 1, wherein the one or more processors being configured to determine the position of the ultrasound probe comprises the one or more processors being configured to detect, based on a machine learning model, visual features associated with the ultrasound probe in the images of the medical environment, and determine the position of the ultrasound probe based on the detected visual features.

9. The system of claim 1, wherein the one or more processors are further configured to:
receive a second ultrasound image of the patient captured using the ultrasound probe;
determine, based on respective visual features of the first ultrasound image and the second ultrasound image detected by a machine learning model, that the first ultrasound image is substantially similar to the second ultrasound image; and
provide an indication that the first ultrasound image and the second ultrasound image are duplicative of each other.

10. The system of claim 1, wherein the one or more processors are further configured to detect, based on a machine learning model, a medical abnormality in the first ultrasound image, and provide an indication of the detected medical abnormality.

11. The system of claim 1, wherein the at least one sensing device is configured to be installed on the ultrasound machine or from a ceiling of the medical environment.

12. The system of claim 1, wherein the one or more processors being configured to determine the position of the ultrasound probe comprises the one or more processors being configured to determine the position of the ultrasound probe relative to the patient's body.

13. A method, comprising:
capturing images of a medical environment, wherein the medical environment includes a patient and an ultrasound machine, and wherein the ultrasound machine includes an ultrasound probe;
obtaining a three-dimensional (3D) human model of the patient, wherein the 3D human model indicates at least a pose and a shape of the patient's body;
receiving a first ultrasound image of the patient captured using the ultrasound probe;
determining, based on the images of the medical environment, a position of the ultrasound probe;
aligning the first ultrasound image with the 3D human model based on at least the position of the ultrasound probe; and
generating a visual representation that shows the alignment of the first ultrasound image and the 3D human model.

14. The method of claim 13, wherein the visual representation includes a 3D body contour of the patient, and wherein the method further comprises filling a first inside portion of the 3D body contour with the first ultrasound image based on the alignment of the first ultrasound image and the 3D human model.

15. The method of claim 14, further comprising:
receiving a second ultrasound image of the patient captured using the ultrasound probe;
aligning the second ultrasound image with the 3D human model based on at least the position of the ultrasound probe; and
adding the second ultrasound image to the visual representation by filling a second inside portion of the 3D body contour with the second ultrasound image based on the alignment of the second ultrasound image and the 3D human model.

16. The method of claim 15, wherein the first and second ultrasound images of the patient are associated with an anatomical structure of the patient, and wherein the method further comprises reconstructing a 3D ultrasound model of the anatomical structure based on at least the first ultrasound image and the second ultrasound image.

17. The method of claim 13, further comprising determining an orientation of the ultrasound probe, wherein the first ultrasound image is aligned with the 3D human model further based on the determined orientation of the ultrasound probe.

18. The method of claim 13, wherein determining the position of the ultrasound probe comprises detecting, in the images of the medical environment, a marker associated with the ultrasound probe and determining the position of the ultrasound probe based on the detected marker.

19. The method of claim 13, determining the position of the ultrasound probe comprises detecting, using a machine learning model, visual features associated with the ultrasound probe in the images of the medical environment, and determining the position of the ultrasound probe based on the detected visual features.

20. The method of claim 13, further comprising:
receiving a second ultrasound image of the patient captured using the ultrasound probe;
determining, based on respective visual features of the first ultrasound image and the second ultrasound image detected by a machine learning model, that the first ultrasound image is substantially similar to the second ultrasound image; and
providing an indication that the first ultrasound image and the second ultrasound image are duplicative of each other.
